(19) United States
(12) Patent Application Publication
(10) Pub. No.: US 2007/0167739 A1
(43) Pub. Date: Jul. 19, 2007

(54) Internally directed imaging and tracking system

(76) Inventor: Rodney W. Salo, Fridley, MN (US)

Correspondence Address: SCHWEGMAN, LUNDBERG, WOESSNER & KLUTH, P.A., P.O. BOX 2938, MINNEAPOLIS, MN, 55402, US

(21) Appl. No.: 11/297,658

(22) Filed: Dec. 7, 2005

(51) Int. Cl. A61B 5/05

(52) U.S. Cl. 600/424

(57) ABSTRACT

A system and methods for enhancing non-invasive imaging and tracking by providing an internal marker detectable by an external imaging system is disclosed. The marker may be active or passive by either generating or reflecting energy. The imaging system may be utilized to optimize imaging parameters to compensate for aberrations in the detected energy based on a known location of the marker, thereby correcting the acquired image. The system may also track the marker automatically, thereby tracking the desired image. This permits, for example, continuous ultrasonic imaging of the heart without requiring constant operator attention.

### TECHNICAL FIELD

The present system and methods relate generally to an implantable medical device, and particularly, but not by way of limitation, to such a device that comprises markers suitable for imaging and an automated methodology for imaging.

### BACKGROUND

The acquisition of cardiac images by 2D ultrasound generally requires a trained sonographer who is able to accurately position the ultrasound transducer on the patient's thorax and orient the ultrasound beam in three dimensions to scan the plane of interest. This is done using landmarks within or near the heart that are often not easily recognizable to the untrained observer. The accuracy of serial measurements from these images, whether made over minutes or years, is dependent upon obtaining the same views multiple times.

For this and other reasons, there is a need for a system that improves the ease and accuracy of transducer placement and the reproducibility of image capture.

### SUMMARY

According to one aspect of the invention, there is provided a system for an internally directed imaging and tracking system for enhanced non-invasive imaging by providing an internal marker detectable by an external imaging system. This may be an active or passive marker that either generates or reflects energy. The external system may then optimize imaging parameters to compensate for aberrations in the detected energy based on a priori knowledge of the marker, thereby also correcting the acquired image.

The system also may automatically track the marker (which is at a known location on, for example, a human heart), thereby tracking the desired image. This can permit, by way of non-limiting example only, continuous ultrasonic imaging of the heart without requiring continuous operator adjustment.

In an embodiment of the system, the system can be used to improve ultrasonic communication between internal and external components by automatically positioning and controlling the focus of an external ultrasonic energy transducer onto the internal component. This can be done by externally processing the reflected energy or by communicating with the internal component that is monitoring the received external energy.

In another embodiment, the system optimizes the transfer of energy between internal and external components. The system can also be applied to transfer external energy to internal components, like to recharge an internal battery or to otherwise provide energy for the operation of internal components.

In a further embodiment, the system is applicable to non-imaging situations in which ultrasonic or other forms of energy are transferred between internal and external components. This embodiment permits improvements in the efficiency of the energy transfer by minimizing the spread of the energy to other regions and permitting operation at lower overall energy. This embodiment can also be useful in the case of an implanted sensor or medical device that is communicating with an external system and/or being powered by an external system.

The various embodiments described above are provided by way of illustration only and should not be construed to limit the invention. Those skilled in the art will readily recognize various modifications and changes that may be made to the present invention without following the example embodiments and applications illustrated and described herein, and without departing from the true spirit and scope of the present invention, which is set forth in the following claims.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

FIG. 1 is a schematic/block diagram illustrating one embodiment of the system for an internally directed imaging and tracking system for an implantable medical device or its components.

FIG. 2 is a schematic/block diagram illustrating another embodiment of a system for an internally directed imaging and tracking system for an implantable medical device or its components.

FIG. 3 is a schematic/block diagram illustrating an embodiment of an implantable medical device suitably adapted for an internally directed imaging and tracking system.

### DETAILED DESCRIPTION

In the following detailed description, reference is made to the accompanying drawings that form a part hereof, and in which is shown by way of illustration specific embodiments or examples. These embodiments may be combined, other embodiments may be utilized, and structural, logical, and electrical changes may be made without departing from the spirit and scope of the present invention. The following detailed description is, therefore, not to be taken in a limiting sense, and the scope of the present invention is defined by the appended claims and their equivalents.

The present system is described with respect to a system that is adapted for internally directed imaging and tracking of an implantable medical device or its component parts to optimize and manage the positioning of the device or component parts at the time of implantation and/or after implantation.

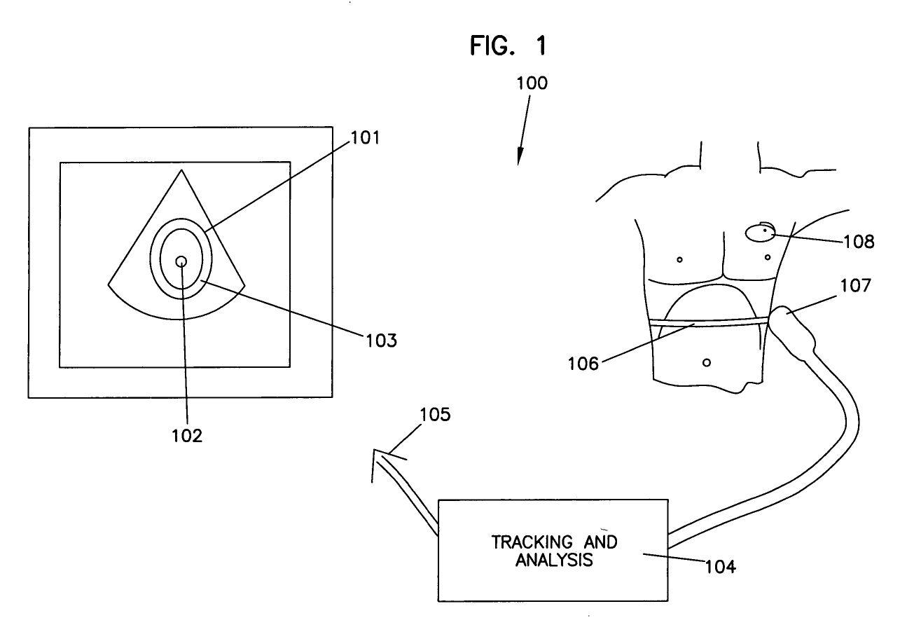

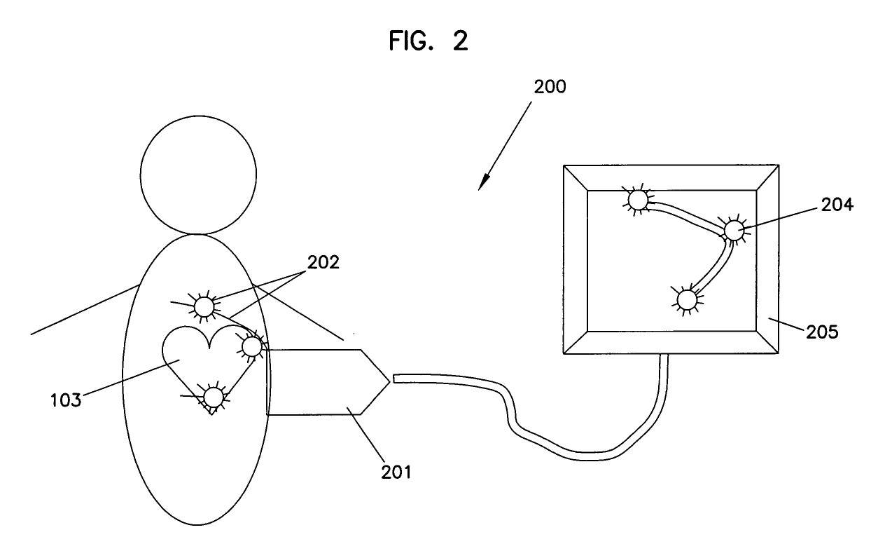

As shown in FIGS. 1 and 2, the systems 100, 200 include an ultrasonic or other energy source 101, 201 sufficient to energize a marker 102 (system 100) or plurality of markers 202 (system 200) positioned on the device and/or its component parts. Using relative positioning techniques, the relative locations of the markers to the implant sites can be determined to accuracies of clinical significance. Thus, the relative positions of the markers and the physical dimensions of the implant site can be determined. By way of non-limiting example only, the implant site can include the dimensions or axes of a mammalian heart 103. In this way, the system can provide appropriate feedback to a clinician on the position of the device or the implant site.

The term “clinician,” as used herein, can mean a physician, physician assistant (PA), nurse, medical technologist, or any other patient health care provider. The term “operator,” as used herein, generally refers to a sonographer, echocardiographer or other clinician skilled in sonography or ultrasound technologies.

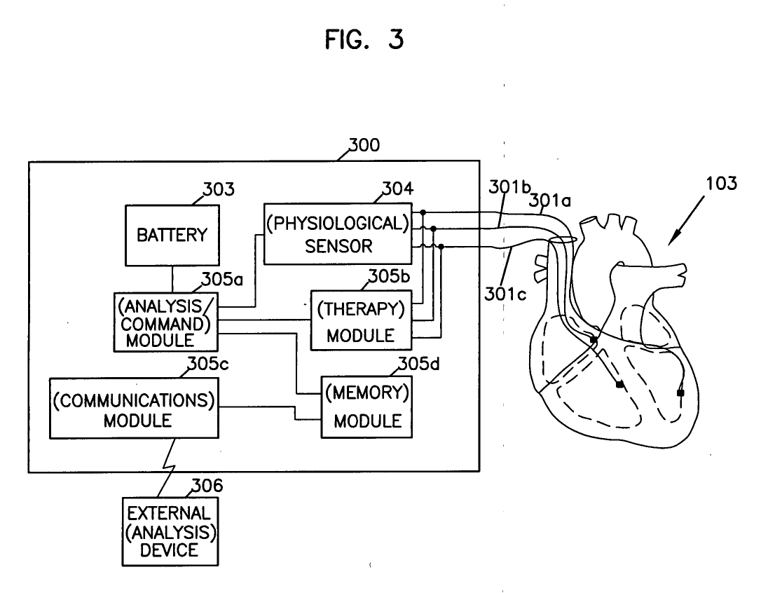

In an embodiment where the component parts of implantable medical devices 108, 300, like a pulse generator or defibrillator, are marked for external imaging. For example, device 300 shown in FIG. 3 includes leads 301a-c that may be marked. A suitable implantable medical device such as device 300 may include a battery 303, at least one sensor 304 and a plurality of modules 305a-d, said modules typically comprising an analysis/control module, a therapy module, a communications module, and a memory module. By way of non-limiting example only, the implanted lead 202 may include ultrasound markers 204. By way of further non-limiting example only, at least three leads of the device may be marked or a single lead may be marked in three locations, and the markers may comprise an energizable piezoelectric or ultrasonic crystal (ultrasonic crystals are usually piezoelectric although other technologies exist). Crystalline markers may be further adapted to resonate or ping an energy signal on command by the implant to further enhance precise location of the marker. As shown in FIG. 2, the markers 202, either piezoelectric or using different ultrasound technologies, may be electronically oriented in relation to an ultrasonic transducer 201 in such a manner that a geometric plane 204 within the heart 203 is well defined.

The ultrasound image 205 is analyzed for sufficient intensity from the markers and feedback given to the operator to find an optimal position/orientation. The image analysis module 104 may be accomplished using an external device 306 such as programmer, or within the implantable device 300. Once an optimal position has been initially established, the system 200 triangulates to remember that position as a baseline for later follow-ups. Ultrasonic tracking systems of the type described herein may also be integrated with an implantable medical device programmer or be adapted to communicate with a programmer.

In an embodiment where the markers comprise a piezoelectric crystal, the crystal may be energized by ultrasound, which in turn may generate electricity to charge a battery of an implantable medical device like a pacemaker. In this embodiment, the precise imaging aspect of the crystal allows for targeted focus of the energy beam to maximize the efficiency of the charging system.

In another embodiment using a crystal, the crystal may be inserted into an animate body like a tumor and then energized, thereby generating heat. In this embodiment, not only can the location of the crystal be readily determined, but it can also serve as a therapeutic agent to apply pinpoint heat to a tumor and destroy it.

In a further embodiment, the positions of the markers are automatically analyzed and tracked using a tracking and analysis module 104 that generates image 105 and verbal or visual feedback given to the clinician to maintain acceptable transducer position. Feedback also can be supplied in the form of data. By way of non-limiting example only, the energy received by a crystal could be transmitted or conveyed to the clinician allowing her to focus or aim the ultrasonic beam in the optimum direction and monitor the energy transfer between the ultrasonic beam and the crystal. In one example, tracking and analysis module 104 is a programmer.

Using visual feedback, the displayed images 105, 205 may be adjusted to maintain marker position on the screen as the heart moves during a cardiac cycle or to correct for small movements in the transducer.

In another embodiment, the transducer and/or beam orientation is controlled by the system to maintain marker position. Such an embodiment may require a mechanized transducer holder for large movements and the ability to control the phased array for smaller movements. A preferred embodiment is adapted to use an implantable medical device or at least one of its component parts to automate the positioning of the external ultrasound transducer to acquire a cardiac image with improved accuracy and reproducibility. When detecting smaller movements is the clinical goal, an embodiment may comprise a belt 106 that would be placed around the torso of a patient that would hold the transducer 107 in a fixed position, yet allow the angle of the beam to change in response to the analyzed positions of the markers.

In its various embodiments, the system permits cardiac resynchronization therapy (“CRT”) optimization by echo through the continuous acquisition of echo data (Doppler or 2D) during a pacing protocol without requiring a specially trained operator. CRT implantable medical devices improve the mammalian heart's pumping ability by delivering small electrical impulses that help synchronize contractions of the chambers of the heart. The left ventricle is the heart's main pumping chamber, and its ability to pump blood is enhanced when the muscular walls contract synchronously. In addition, CRT devices monitor the heart for potentially fatal rhythms. If such a rhythm is detected, an implantable medical device like a defibrillator can deliver an electrical shock, which restores normal heart rhythm and prevents sudden cardiac death.

In yet another embodiment, the system provides a method of internally directed imaging and tracking of an implanted medical device comprising a plurality of ultrasonic markers. A method allows for directing ultrasonic energy using an ultrasonic transducer on the implanted device to analyze the positions of the markers and determine a geometric plane comprising the positions of at least three markers of the implanted device. After establishing this baseline position, the transducer may be automatically positioned relative the determined geometric plane to create a stable image of the implanted medical device relative the site of implantation. This positioning information can be used to further direct ultrasonic energy onto the implantable medical device to maximize CRT.

The embodiments disclosed herein do not require trained sonographers or echocardiographers for CRT optimization. For example, a nurse or EP may use the system with only minimal training in sonography or echocardiography. Such optimization can occur at the time of implant and/or during a follow-up procedure. With good image quality and stability, optimization can be automated, thereby reducing follow-up time while improving patient outcome.

The disclosed embodiments may also determine and confirm lead placement and location by echo at the initial implant stage or during a follow-up procedure to check for lead dislodgement or movement. The system may also be used with a battery powered remote sensor to optimize transducer orientation for ultrasonic battery charging.

It is to be understood that the above description is intended to be illustrative, and not restrictive. For example, the above-described embodiments may be used in combination with each other. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including,” “includes” and “in which” are used as the plain-English equivalents of the respective terms “comprising,” “comprises” and “wherein.”

What is claimed is:

1. A system for internally directed imaging and tracking comprising:
a. an implant comprising at least one marker;
b. an ultrasonic transducer adapted for ultrasonic imaging;
c. a microprocessor-based analysis module adapted to analyze an ultrasonic image, a position of a marker and deviations in marker position;
d. a positioning feedback module coupled to the analysis module to position the ultrasonic transducer; and
e. an image orientation correction module adapted to use position information.

2. The system of claim 1, wherein the implant comprises a plurality of markers.

3. The system of claim 1, wherein the marker comprises an active or passive marker.

4. The system of claim 1, wherein the marker comprises an energy generating or reflective marker.

5. The system of claim 4, wherein the analysis module is adapted to externally process the energy of the marker or communicate with the marked implant.

6. The system of claim 1, wherein the marker comprises an ultrasonic energizable marker.

7. The system of claim 1, wherein the marker comprises an ultrasonic energizable crystal marker adapted to resonate energy upon command by the implant.

8. The system of claim 7, wherein the energizable crystal marker comprises a piezoelectric crystal marker.

9. The system of claim 1, wherein the marked implant includes a battery.

10. The system of claim 1, wherein the ultrasonic transducer transfers energy to a marked implant that is adapted to be implanted in an animate or inanimate body.

11. The system of claim 10, wherein the animate body includes a tumor.

12. The system of claim 10, wherein the transferred energy creates heat.

13. The system of claim 1, wherein the positioning feedback module provides verbal feedback.

14. The system of claim 1, wherein the positioning feedback module provides visual feedback.

15. The system of claim 1, wherein the positioning feedback module provides data feedback.

16. The system of claim 1, wherein the analysis module analyzes the ultrasonic image controls the focus of ultrasonic transduction onto the marked implant.

17. The system of claim 1, wherein the analysis module controls focus of ultrasonic transduction onto the marked implant.

18. A system for internally directed imaging and tracking comprising:
a. an implantable medical device comprising a plurality of markers and a battery;
b. an ultrasonic transducer adapted for ultrasonic imaging and to transfer energy to the implant to charge the battery;
c. a microprocessor-based analysis module adapted to analyze an ultrasonic image and a position of a marker;
d. a positioning feedback module coupled to the analysis module to position the ultrasonic transducer; and
e. an image orientation correction module adapted to use position information to automatically position the transducer to provide continuous ultrasonic imaging of the image.

19. The system of claim 18, wherein the implantable medical device comprises one or more of the following: a pulse generator, a defibrillator, a stent, a catheter or a lead.

20. A method of internally directed imaging and tracking, comprising:
a. implanting an implantable medical device comprising a plurality of ultrasonic markers in vivo;
b. directing ultrasonic energy using an ultrasonic transducer on the implantable medical device;
c. analyzing the positions of the markers to determine at least one geometric plane comprising the positions of at least three markers of the implantable medical device;
d. automatically positioning the directed ultrasonic energy relative a geometric plane of markers;
e. creating an image of the implantable medical device relative the implant site; and
f. further directing ultrasonic energy onto the implantable medical device to maximize cardiac resynchronization therapy.
